(19) United States
(12) Patent Application Publication
(10) Pub. No.: US 2025/0006342 A1
(43) Pub. Date: Jan. 2, 2025

(54) MENTAL HEALTH INTERVENTION USING A VIRTUAL ENVIRONMENT

(71) Applicants: VANDERBILT UNIVERSITY, Nashville, TN (US); INNERWORLD, INC., Nashville, TN (US)

(72) Inventors: Noah ROBINSON, Nashville, TN (US); Callum GOLDS, Nashville, TN (US); Tanner NETTERVILLE, Nashville, TN (US)

(73) Assignees: VANDERBILT UNIVERSITY, Nashville, TN (US); INNERWORLD, INC., Nashville, TN (US)

(21) Appl. No.: 18/698,611

(22) PCT Filed: Oct. 4, 2022
(86) PCT No.: PCT/US2022/045651

§ 371 Date: Apr. 4, 2024

Related U.S. Application Data
(60) Provisional application No. 63/251,844, filed on Oct. 4, 2021.

(51) Int. Cl. G16H 20/70; G06F 3/01; G06T 13/40; G10L 15/26; G16H 10/20; G16H 40/67; G16H 50/20

(52) U.S. Cl. CPC G16H 20/70; G06F 3/013; G06F 3/017; G06T 13/40; G10L 15/26; G16H 10/20; G16H 40/67; G16H 50/20

(57) ABSTRACT

Systems and methods are provided for providing mental health intervention using a virtual environment. A spoken communication from a first user is conveyed to a second user in a virtual environment. Data about the first user is collected as the first user interacts with the second user. The collected data includes tone information from the spoken communication. A clinical parameter representing a mental state of the first user is determined from the collected data at a machine learning model. A prompt is provided to the second user, representing one of a suggested phrase, a suggested sentence, and a suggested topic of conversation, according to the determined clinical parameter.

### RELATED APPLICATIONS

This application claims the benefit of U.S. Provisional Application Ser. No. 63/251,844, filed Oct. 4, 2021. This provisional application is hereby incorporated by reference in its entirety for all purposes.

### TECHNICAL FIELD

This invention relates to medical information systems, and more particularly, to mental health intervention using a virtual environment.

### BACKGROUND

While awareness of mental health concerns has increased over the last decade, the resources for addressing these concerns have not, placing significant pressure on mental health professionals. Training a clinician is a lengthy process, it is difficult to provide enough trained and licensed therapists to service a rising population in need of intervention. The recent increase in the use of video teleconferencing services has been used by some therapists to service a larger number of patients, but this has been insufficient to address the need for intervention services.

### SUMMARY

In accordance with one example, a method is provided for providing mental health intervention using a virtual environment. A spoken communication from a first user is conveyed to a second user in a virtual environment. Data about the first user is collected as the first user interacts with the second user. The collected data includes tone information from the spoken communication. A clinical parameter representing a mental state of the first user is determined from the collected data at a machine learning model. A prompt is provided to the second user, representing one of a suggested phrase, a suggested sentence, and a suggested topic of conversation, according to the determined clinical parameter.

In accordance with another example, a system includes an input device that allows a first user to interact with a second user in a virtual environment hosted on a server. The input device includes a microphone to allow spoken communication from the first user to the second user. The server includes a network interface, a processor, and a non-transitory computer medium that stores executable instructions that, when executed by the processor, provide a helper support component that provides a prompt to the second user, representing one of a suggested phrase, a suggested sentence, and a suggested topic of conversation, according to a determined clinical parameter representing a mental state of the first user. The helper support system includes a data collection component that collects data about the first user as the first user interacts with the second user. The collected data including tone information from the spoken communication. A machine learning model determines the clinical parameter from the collected data.

In accordance with a further example, a method is provided for providing mental health intervention using a virtual environment. A spoken communication from a first user is conveyed to a second user in a virtual environment. Data about the first user is collected as the first user interacts with the second user. The collected data includes tone information from the spoken communication. A clinical parameter representing a mental state of the first user is determined from the collected data at a machine learning model. A prompt is provided to the second user, representing one of a suggested phrase and a suggested sentence, according to the determined clinical parameter. A response is received from the second user selecting the one of the suggested phrase and the suggested sentence, and audio representing the one of the suggested phrase and the suggested sentence is played to the first user.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 illustrates an example of a system for providing mental health intervention in a virtual environment;

FIG. 2 illustrates another example of a system for providing mental health intervention in a virtual environment;

FIG. 3 illustrates another method for providing mental health intervention in a virtual environment;

FIG. 4 illustrates another method for providing mental health intervention in a virtual environment; and

FIG. 5 is a schematic block diagram illustrating an exemplary system of hardware components capable of implementing examples of the systems and methods disclosed herein.

### DETAILED DESCRIPTION

In the context of the present disclosure, the singular forms “a,” “an” and “the” can also include the plural forms, unless the context clearly indicates otherwise. The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “substantially identical” or “substantially equal” refers to articles or metrics that are identical other than manufacturing or calibration tolerances.

The systems and methods described herein provide a virtual reality application that delivers live, synchronous cognitive behavioral intervention within a virtual environment. It can be accessed via an immersive virtual reality device or in 2-D mode through desktop computers or mobile phones. The platform provides a virtual environment in which users can interact with one another anonymously or pseudoanonymously using avatars designed or selected by the user. As users interact with the therapeutic environment, they can gain experience points in a manner similar to a massive multiplayer online environment such as World of Warcraft. These points can increase user levels that are displayed for others to see. Psychometric data and metadata can be used to affect a user's level or role.

In additional to allowing individuals with various mental health issues to support one another, the platform can collect both objective behavioral data, such as speech content, tone, movement data, reaction times, and various metadata, as well as self-reported data provided by the user, for example, via surveys. This data can be provided to clinicians to individualize the treatment provided to these patients. In the context of the virtual environment, a “helpee “is a user of the platform who is receiving services, and a “helper” is a peer (someone also struggling with a mental health disorder), or lay counselor (someone who has received training) that provides help to helpees. Any intervention delivered by a helper cannot be considered “therapy” since it is not done by a licensed professional, although it will be appreciated that this intervention is intended to reduce the impact of the mental health disorder on the individual. A group is a gathering of individuals that are receiving interventions from a trained person. This person could be a therapist or untrained lay counselor, and the interventions can include, for example, peer groups, therapy groups, and dyads.

The platform can also enable other researchers to upload their own environments and recruit participants to participate in the environments. Researchers can have access to specific surveys they upload, as well as a standardized set of psychometric data. Researchers will obtain consent from participants. Additionally, prospective participants among registered users can be notified of eligibility if they meet criteria for researchers. They can receive a notification and sign an IRB consent to participate in the experiment, which could also include providing access to data that has been collected on them throughout their experience in the application. This dynamic data sharing and research participation will allow the system to provide a large-scale clinical research platform.

FIG. 1 illustrates an example of a system 100 for providing mental health intervention in a virtual environment. The system 100 provides a virtual environment for helpers to engage with helpees in a controlled, supervised environment. In particular, verbal interactions between helpers and helpees can be transcribed via a voice recognition system to provide a text transcript of the interactions in real time to a licensed professional, allowing a professional to supervise multiple interactions simultaneously. Further, data collected from helpees can be used to advise helpers in their interactions, for example, by providing suggested phrases, sentences, or topics of conversation. Finally, the environment can utilize an experience system to reward users for engaging in activities, encouraging activity on the system 100 via a gamification strategy. By allowing and encouraging users to interact in a supervised environment, a single professional can manage interventions for a number of individuals, allowing for efficient use of the professional's time.

The system 100 includes at least one input device 102 that allows a first user to interact, via a client application 104, with a second user in a virtual environment hosted on a server 110. It will be appreciated that, where the client application 104 associated with the input devices is executed on a desktop or laptop computer, the input devices can include any or all of a keyboard, mouse, display, microphone, and speaker. Alternatively, one or more of virtual reality goggles, motion sensors, and joysticks can be used to provide a more immersive environment. Where the client application 104 associated with the input devices is executed on a mobile device, the input devices can include any or all of a touchscreen, microphone, and speaker. To supplement any of these general arrangements, wearable or portable sensors can be included to monitor one or more physiological parameters of the first user. In general, the input devices will include a microphone to allow spoken communication from the first user to the second user.

The server 110 includes a network interface 112, a processor 114, and a non-transitory computer readable medium 116 that stores executable instructions that, when executed by the processor, provide a helper support component 120. The helper support component 120 provides a prompt to the second user, representing one of a suggested phrase, a suggested sentence, and a suggested topic of conversation, according to a determined clinical parameter representing a mental state of the first user. The helper support system 120 includes a data collection component 122 that collects data about the first user as the first user interacts with the second user including at least tone information from the spoken communication. It will be appreciated that by “tone information,” it is meant both the semantic content of the communication as well as audio information extracted from the spoken communication, which can be used to determine an affect of the first user. The collected data can also include, for example, physiological data from sensors worn or carried by the first user, data from motion sensors worn or carried by the first user that can be used to derive a position of the user in the virtual environment or a target of a gaze of the user within the virtual environment, and metadata representing the activity of the user in the virtual environment.

A machine learning model 124 determines the clinical parameter from the collected data. In one example, the clinical parameter represents a specific psychological issue associated with the first patient, such as anxiety, depression, addiction, stress, or grief. In another example, the clinical parameter represents an intervention expected to be helpful for the first user, such as a specific support group or helper. In a further example, the clinical parameter represents the existence or progression of a mental disorder associated with the first user. In a still further example, the clinical parameter is a categorical parameter representing the suggested phrase, sentence, or topic of conversation directly. In examples in which the clinical parameter is not the suggested phrase, sentence, or topic of conversation, a rule-based expert system can be used to select the suggested phrase, sentence, or topic of conversation from the clinical parameter. In a straightforward example, when the clinical parameter indicates that the first user is experiencing a particular psychological issue or emotion (e.g., grief), the expert system can select an appropriate phrase, sentence, or topic of conversation from a library of such phrases, sentences, and topics based on the clinical parameter and profile data for the first user. In one example, the helper support component 120 includes a text-to-speech component (not shown) that plays audio representing the one of a suggested phrase and the suggested sentence to a first user if the suggested phrase is selected by the second user.

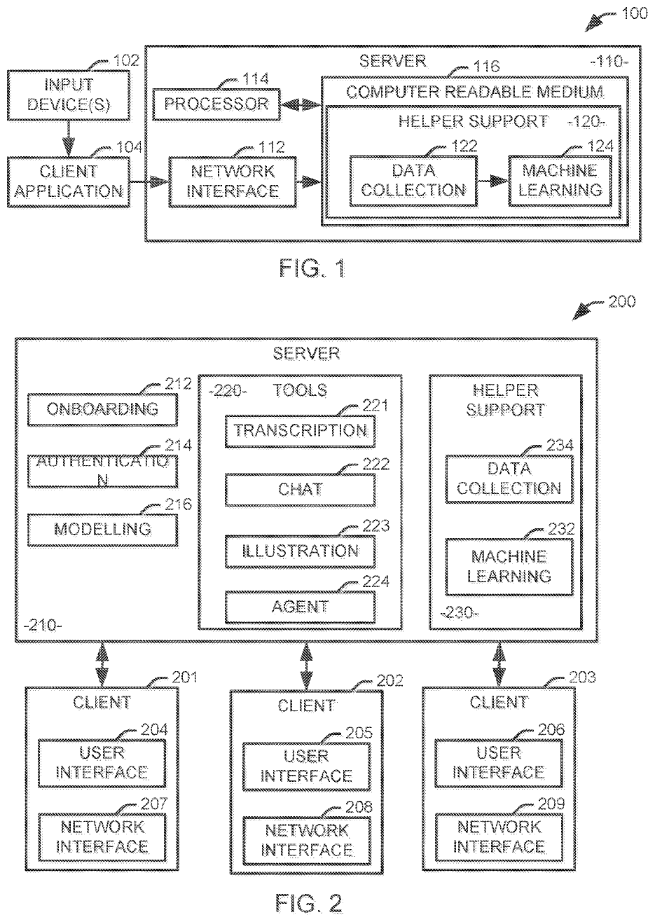

FIG. 2 illustrates another example of a system 200 for providing mental health intervention in a virtual environment. The system 200 includes a plurality of client devices 201-203 and a server 210. Each client device 201-203 can be implemented as a personal computing device, such as a desktop computer, a laptop computer, a tablet, a smart phone, or a video game console. Each client device 201-203 includes a user interface 204-206 that allows the client device to receive input from a user and provide at least visual and auditory data to the user from the server. In one implementation, the user interface 204-206 communicates with a touchscreen, speaker, and microphone to receive data from the user and convey data from the server to the user. Alternatively, the touchscreen can be replaced with a keyboard, mouse, and standard display, or with a set of virtual reality goggles and motion detecting sensors in a handheld or worn article (e.g., a glove). It will be appreciated that the virtual environment can be rendered in three-dimensions when the virtual reality goggles and sensors are used to navigate the environment and in two-dimensions when a standard display or touchscreen is used. The network interface 207-209 allows each client device to communicate with the server via an Internet connection. The server 210 can be implemented as any appropriate combination of hardware and software elements for hosting the virtual environment platform. In one implementation, the server 210 is implemented as a cloud server that can be dynamically scaled up and down depending on the number of instances required for usage.

The server 210 can store instructions for implementing an onboarding component 212 that is configured to receive information from users to register users with the system 200. The onboarding system 212 can receive a registration code from a user seeking to register with the system that identifies a referral source for the user, if any, and allows the user to begin a screening process. The screening process can be automated or semi-automated, with the user provided with a series of questions and other prompts, either in written form or verbally within the virtual environment. The user's answers to the questions, as well as behavioral data collected during the screening, can be collected and used to determine if the user is an appropriate candidate for enrollment in the peer support groups managed by the system 200. For example, the screening can be used to identify users who are potentially a risk to themselves or others. In one implementation, other users can be trained to host the intake events within the virtual reality environment. It will be appreciated that all information collected from the user will be maintained in encrypted form on the server 200, with access to this information regulated in accordance with the Health Insurance Portability and Accountability Act of 1996 (HIPPA).

Once the user has been registered with the system 200, appropriate credentials can be selected by the user or provided by the onboarding component 212 and registered with an authentication component 214. The credentials can include, for example, an identifier, such as a username or identification number, along with a password that is stored in a hashed format at the authentication component. A password entered by the user can be hashed and compared to the stored hash to authenticate the user. It will be appreciated that users can have varying levels of access for the system. For example, new patients may have a first level of access, more experienced patients who have qualified as “peer helpers” may have a second level of access, clinicians may have a third level of access, and administrators may have a fourth level of access. Each level of access can include various levels of control over the virtual environment, access to tools associated with the virtual environment, and stored data collected from the environment. Upon logging in, the user appears in an “offline home,” a non-networked environment that can include can have personalized elements, such as pictures of loved ones, tools that have been used in the app, and psychometric data displayed. The online home may also contain levels, badges or other designations earned through gamification, as will be discussed below. In one implementation, external researchers can be given a level of access that allows them to upload their own environments and recruit participants to participate in the environments.

A modelling component 216 stores and instantiates the various three-dimensional models used to construct the virtual environment. The modelling component 216 can include templates and models for constructing avatars and objects within the virtual environment as well as specific models generated by users and saved at the server. It will be appreciated that saved models can be associated with one or more user accounts, with certain objects available to all users of a given access level. The stored objects can include various environments that have been constructed by users or by administrators, interfaces for various tools that can be used in the virtual environment, and objects for constructing or decorating existing environments.

The server 200 can also store a plurality of tools 220 that can be employed by helpers and helpees. For example, a transcription tool 221 can be employed to maintain a text transcript of voice data during conversations between users. In one implementation, the voice data is transmitted using the Voice Over Internet Protocol (VIOP). This can be useful for monitoring interaction between users to detect and discourage unhelpful or malicious behavior, for example, by screening the text for key words. The stored text can also be provided to clinicians in a chat window to guide therapeutic actions, as well as to ensure that helpers who are not licensed therapists are supervised by a clinician. Since the voice data is transcribed in real-time and can be immediately used to populate a chat window, this tool can also be useful for facilitating communication with individuals with hearing impairment. A chat tool 222 can allow users to interact via text instead of voice. This can be used for one-to-one interactions or as a group messaging system.

An illustration tool 223 can be used to provide visual data to other users, either through freeform drawing in two or three dimensions or via placement and annotation of existing models. Items created using the illustration tool 223 can be placed and moved in real time. Users can gather around the tools, view them within a three-dimensional space, and dynamically interact with the tools. Illustrations created with these tools are recorded and can be displayed to users in an offline home area. One example of an existing model is a cognitive behavior model that allows a user to label a situation and record thoughts, feelings, physiological reactions, and behaviors associated with the situation via interaction with the model. For example, a user can click on the word “Feelings,” and a list of feelings will appear from which the user can select, with a slider under each feeling for the user to rate the feeling. Similarly, a user can click on “thoughts” “behaviors” and “physiology” at which point a text box pops up in which the user can record thoughts either via text to speech, in which case emotional tone analysis is collected, or via a virtual/physical keyboard input device.

An agent tool 224 can be employed by helpers to facilities communication with helpees, particularly when the helper is conversing with multiple helpees. An agent is an avatar capable of automated or semi-automated operation, often appearing the same way as the avatar of a real user, that can play pre-recorded speech and reproduce recorded motions, such as gesturing. It will be appreciated that an agent can be fully controlled by a human being, controlled by a human being with some automated behaviors, or be fully automated. The agent tool 224 can include a text-to-speech component that translates typed chats into speech for a given avatar. It may also translate text, in real-time or previously stored text, while using machine learning to engage in gestures that seem realistic. This can allow a single helper to operate multiple avatars at a same time. Agents can be used to potentially lead groups. The machine learning algorithms for agents will be tested with live helpers, who will suggest dynamic prompts and feed the algorithms to improve them over time, with the goal of leading group sessions and individual sessions with artificial intelligence agents.

A helper support component 230 utilizes a machine learning model 232 to assign a clinical parameter representing a mental state of a patient. For example, the collected data can be used to identify specific issues that a person is dealing with (e.g., anxiety, depression, addiction, stress, or grief). The clinical parameter can also dynamically suggest the type of support group a user could attend. In such a case, the clinical parameter is a categorical parameter with values representing various support groups. Alternatively, the clinical parameter can be a categorical parameter representing prompts provided to a helper. These prompts can suggest a tool for the helper to use, a suggested topic of conversation, or a phrase/question that could be helpful for them to add. The system 200 monitors real-time text data and also pulls from an existing database of possible tools, phrases, or questions. Helper engagement with the suggestion system, such as what suggestions the helpers use or do not use, can be data inputs to the machine learning model 232 for retraining the model and/or adjusting the available values of the clinical parameter. Additionally or alternatively, the clinical parameter can also represent types of psychopathologies or the actual or expected efficacy of a given treatment.

A data collection component 234 allows for data to be collected from participants prior to or during groups. This data can include self-reported subjective data from surveys, movement data captured by the input device, a location within the virtual world, a location at which the person is looking in the environment (measured through head movement or approximated by where the field of view is pointed), speech data, and metadata (e.g., how often and how long someone logs in). Physiological data can be monitored through wearables and can include heartrate variability, galvanic skin response, and other indicators. These are collected with a timestamp that can be correlated to the user's actions within the virtual environment. All data is used as an input mechanism to improve treatment and progress tracking abilities. This data can also be used to prompt surveys. For example, if a user has not logged in for a certain period of time, they can be prompted with a scale that measures perceived loneliness. In addition, helpers can assign surveys to helpees to gather information about psychological states. The collected data can also be used to recommend particular environments or support groups to a user.

In addition to its use in the machine learning model 232 data can be displayed in real time, both in the 3D environment and chat windows or a 2-D dashboard to helpers as they interact with helpees. This data can help to show a helper how the helpee feels in the moment, what issues to focus on, how the helpee's symptoms have changed longitudinally, and any other indicators of treatment progress. Data can be hidden depending on the user's permission level. This system can also receive inputs from a real person. A trained person can monitor a chat window and can drag/drop suggested prompts or tools into the window, which appear to the helper leading the group and are only visible to the helper. The helper can select the suggestion or ignore it.

The machine learning model 232 can utilize one or more pattern recognition algorithms, implemented, for example, as classification and regression models, each of which analyze provided data to assign a clinical parameter to the user. It will be appreciated that the clinical parameter can be categorical or continuous. In some models, digital information representing audio, video, or images can be provided directly to the machine learning model 232 for analysis. For example, convolutional neural networks can often operate directly on provided chromatic or intensity values for the pixels within an image file or amplitude values within an audio file, such as recorded speech from users. Alternatively, the machine learning model 232 can operate in concert with feature extraction logic that extracts numerical features from one or more data sources for analysis by the machine learning model 232. For example, numerical values can be retrieved from local or remote databases, received and buffered directly from one or more sensors or related systems, calculated from various properties of provided media, or extracted from structured, unstructured, or semi-structured text, such as the text chats between users.

Where multiple classification and regression models are used, the machine learning model 232 can include an arbitration element can be utilized to provide a coherent result from the various algorithms. Depending on the outputs of the various models, the arbitration element can simply select a class from a model having a highest confidence, select a plurality of classes from all models meeting a threshold confidence, select a class via a voting process among the models, or assign a numerical parameter based on the outputs of the multiple models. Alternatively, the arbitration element can itself be implemented as a classification model that receives the outputs of the other models as features and generates one or more output classes for the patient. The classification can also be performed across multiple stages. In one example, an a priori probability can be determined for a clinical parameter without the one or more values representing the patient. A second stage of the model can use the one or more values representing the patient, and, optionally, additional values, to generate a value for the clinical parameter. A known performance of the second stage of the machine learning model, for example, defined as values for the specificity and sensitivity of the model, can be used to update the a priori probability given the output of the second stage.

The machine learning model 232, as well as any constituent models, can be trained on training data representing the various classes of interest. For example, in supervised learning models, a set of examples having labels representing a desired output of the machine learning model 232 can be used to train the system. The training process of the machine learning model 232 will vary with its implementation, but training generally involves a statistical aggregation of training data into one or more parameters associated with the output classes. For rule-based models, such as decision trees, domain knowledge, for example, as provided by one or more human experts, can be used in place of or to supplement training data in selecting rules for classifying a user using the extracted features. Any of a variety of techniques can be utilized for the models, including support vector machines, regression models, self-organized maps, k-nearest neighbor classification or regression, fuzzy logic systems, data fusion processes, boosting and bagging methods, rule-based systems, or artificial neural networks.

For example, an SVM classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class and an associated confidence value can be determined for a given input feature vector according to its position in feature space relative to the boundaries. An SVM classifier utilizes a user-specified kernel function to organize training data within a defined feature space. In the most basic implementation, the kernel function can be a radial basis function, although the systems and methods described herein can utilize any of a number of linear or non-linear kernel functions.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for the output classes of the ANN, with each node having an associated value representing a confidence for one of the associated output classes of the classifier.

The classical ANN classifier is fully-connected and feedforward. Convolutional neural networks, however, includes convolutional layers in which nodes from a previous layer are only connected to a subset of the nodes in the convolutional layer. Recurrent neural networks are a class of neural networks in which connections between nodes form a directed graph along a temporal sequence. Unlike a feedforward network, recurrent neural networks can incorporate feedback from states caused by earlier inputs, such that an output of the recurrent neural network for a given input can be a function of not only the input but one or more previous inputs. As an example, Long Short-Term Memory (LSTM) networks are a modified version of recurrent neural networks, which makes it easier to remember past data in memory.

A k-nearest neighbor model populates a feature space with labelled training samples, represented as feature vectors in the feature space. In a classifier model, the training samples are labelled with their associated class, and in a regression model, the training samples are labelled with a value for the dependent variable in the regression. When a new feature vector is provided, a distance metric between the new feature vector and at least a subset of the feature vectors representing the labelled training samples is generated. The labelled training samples are then ranked according to the distance of their feature vectors from the new feature vector, and a number, k, of training samples having the smallest distance from the new feature vector are selected as the nearest neighbors to the new feature vector.

In one example of a classifier model, the class represented by the most labelled training samples in the k nearest neighbors is selected as the class for the new feature vector. In another example, each of the nearest neighbors can be represented by a weight assigned according to their distance from the new feature vector, with the class having the largest aggregate weight assigned to the new feature vector. In a regression model, the dependent variable for the new feature vector can be assigned as the average (e.g., arithmetic mean) of the dependent variables for the k nearest neighbors. As with the classification, this average can be a weighted average using weights assigned according to the distance of the nearest neighbors from the new feature vector. It will be appreciated that k is a metaparameter of the model that is selected according to the specific implementation. The distance metric used to select the nearest neighbors can include a Euclidean distance, a Manhattan distance, or a Mahalanobis distance.

A regression model applies a set of weights to various functions of the extracted features, most commonly linear functions, to provide a continuous result. In general, regression features can be categorical, represented, for example, as zero or one, or continuous. In a logistic regression, the output of the model represents the log odds that the source of the extracted features is a member of a given class. In a binary classification task, these log odds can be used directly as a confidence value for class membership or converted via the logistic function to a probability of class membership given the extracted features.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used, but a continuous parameter can be computed according to a number of decision trees that select a given task. Regardless of the specific model employed, the clinical parameter generated at the machine learning model 232 can be used to select a mental health intervention for a user, provide prompts to a helper, evaluate the efficacy of a mental health intervention, or assist is diagnosis of a user, with the generated clinical parameter available to users at an appropriate level of access to the system.

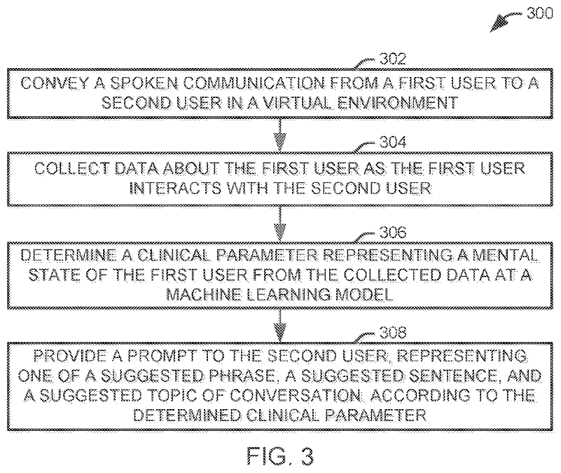

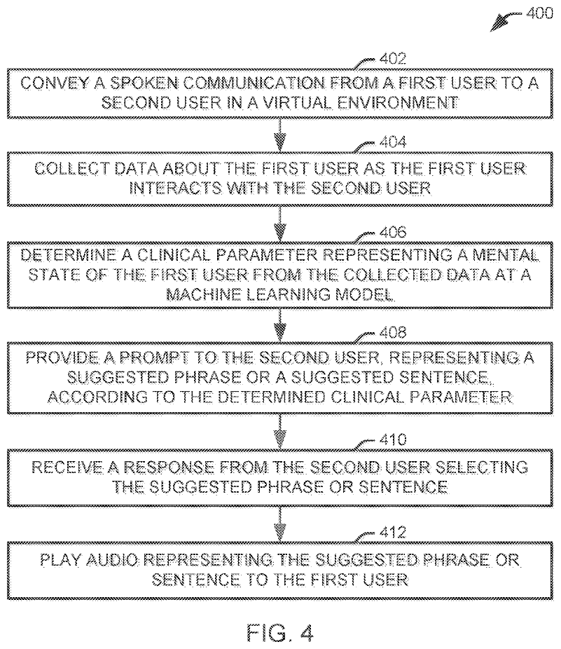

In view of the foregoing structural and functional features described above, example methods will be better appreciated with reference to FIGS. 3 and 4. While, for purposes of simplicity of explanation, the example methods of FIGS. 3 and 4 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement a method in accordance with the invention.

FIG. 3 illustrates one method 300 for providing mental health intervention in a virtual environment. At 302, a spoken communication from a first user is conveyed to a second user in a virtual environment. In one example, speech is recorded at a microphone associated with the first user, sent to a server hosting the virtual environment, and played for anyone interacting with an avatar associated with the first user in the virtual environment. In one example, the spoken communication is provided as audio to the second user. In another example, the spoken communication is provided to a voice recognition system to generate text representing the spoken communication, and the text representing the spoken communication to the second user, for example, in a chat window. Additionally or alternatively, the text representing the spoken communication to a third user who supervises the interaction between the first user and the second user, and in some examples, other interactions between users on the system, such that the third user receives a text representing a spoken communication by users other than the first and second users.

At 304, data about the first user is collected as the first user interacts with the second user, with the collected data including tone information from the spoken communication. It will be appreciated that “tone information” can include the sematic content of the spoken communication as well as data extracted from analysis of the audio file representing the spoken communication. In one example, the collected data includes metadata reflecting a frequency with which the first user accesses the virtual environment and an amount of time for which the first user accesses the virtual environment. Additionally or alternatively, the collected data can include answers to a survey provided to the first user. The collected data can also include motion data from the user representing interaction with the virtual environment as well as physiological data obtained from sensors worn or carried by the first user. In one example, a gamification approach can be used in which the first user has an associated score generated by performing activities within the virtual environment, with the associated score of the first user or a parameter derived from the associated score, such as a “level” being displayed to other users when interacting with the first user. The collected data, as well as activities performed within the virtual environment, can be used in determining the score for the first user.

At 306, a clinical parameter representing a mental state of the first user is determined from the collected data at a machine learning model. In one example, the clinical parameter represents a specific psychological issue associated with the first patient, such as anxiety, depression, addiction, stress, or grief. In another example, the clinical parameter represents an intervention expected to be helpful for the first user, such as a specific support group or helper. In a further example, the clinical parameter represents the existence or progression of a mental disorder associated with the first user. In a still further example, the clinical parameter is a categorical parameter representing the suggested phrase, sentence, or topic of conversation directly.

At 308, a prompt is provided to the second user, representing one of a suggested phrase, a suggested sentence, and a suggested topic of conversation, according to the determined clinical parameter. If the second user selects a suggested phrase or sentence, audio representing the one of the suggested phrase and the suggested sentence can be played to the first user, either via a text-to-speech application or via a stored library of audio for suggested phrases and sentences. In one example, in which the first user and the second user are represented as avatars within the virtual environment, the avatar associated with the second user can be animated to move a mouth to mimic speech and to perform a gesture associated with the suggested phrase and the suggested sentence while playing the audio. For example, the gesture can include a facial expression, change in posture, or movement of the head, hands, and arms that mimic real world body language appropriately during expression of a given sentiment. In one example, the clinical parameter can also be used to select additional surveys for the first user, which can be used in the generation of additional clinical parameters in later interactions.

FIG. 4 illustrates another method 400 for providing mental health intervention in a virtual environment. At 402, a spoken communication from a first user is conveyed to a second user in a virtual environment. In one example, speech is recorded at a microphone associated with the first user, sent to a server hosting the virtual environment, and provided, as text or audio, to anyone interacting with an avatar associated with the first user in the virtual environment. At 404, data about the first user is collected as the first user interacts with the second user, with the collected data including tone information from the spoken communication. The collected data can include any or all of metadata reflecting a frequency with which the first user accesses the virtual environment and an amount of time for which the first user accesses the virtual environment, answers to a survey provided to the first user, motion data from the user representing interaction with the virtual environment, and physiological data obtained from sensors worn or carried by the first user.

At 406, a clinical parameter representing a mental state of the first user is determined from the collected data at a machine learning model. In one example, the clinical parameter represents a specific psychological issue associated with the first patient, such as anxiety, depression, addiction, stress, or grief. In another example, the clinical parameter represents an intervention expected to be helpful for the first user, such as a specific support group or helper. In a further example, the clinical parameter represents the existence or progression of a mental disorder associated with the first user. In a still further example, the clinical parameter is a categorical parameter representing the suggested phrase, sentence, or topic of conversation directly.

At 408, a prompt is provided to the second user, representing one of a suggested phrase and a suggested sentence according to the determined clinical parameter. At 410, a response is received from the second user selecting the one of the suggested phrase and the suggested sentence, and audio representing the one of the suggested phrase and the suggested sentence is played to the first user at 412. In one example, in which the first user and the second user are represented as avatars within the virtual environment, the avatar associated with the second user can be animated to move a mouth to mimic speech and to perform a gesture associated with the suggested phrase and the suggested sentence while playing the audio. For example, the gesture can include a facial expression, change in posture, or movement of the head, hands, and arms that mimic real world body language appropriately during expression of a given sentiment. In one example, the clinical parameter can also be used to select additional surveys for the first user, which can be used in the generation of additional clinical parameters in later interactions.

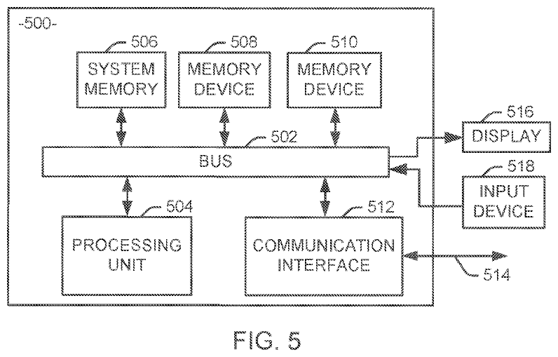

FIG. 5 is a schematic block diagram illustrating an exemplary system 500 of hardware components capable of implementing examples of the systems and methods disclosed herein. The system 500 can include various systems and subsystems. The system 500 can be a personal computer, a laptop computer, a workstation, a computer system, an appliance, an application-specific integrated circuit (ASIC), a server, a server BladeCenter, a server farm, etc.

The system 500 can include a system bus 502, a processing unit 504, a system memory 506, memory devices 508 and 510, a communication interface 512 (e.g., a network interface), a communication link 514, a display 516 (e.g., a video screen), and an input device 518 (e.g., a keyboard, touch screen, and/or a mouse). The system bus 502 can be in communication with the processing unit 504 and the system memory 506. The additional memory devices 508 and 510, such as a hard disk drive, server, standalone database, or other non-volatile memory, can also be in communication with the system bus 502. The system bus 502 interconnects the processing unit 504, the memory devices 506-510, the communication interface 512, the display 516, and the input device 518. In some examples, the system bus 502 also interconnects an additional port (not shown), such as a universal serial bus (USB) port.

The processing unit 504 can be a computing device and can include an application-specific integrated circuit (ASIC). The processing unit 504 executes a set of instructions to implement the operations of examples disclosed herein. The processing unit can include a processing core.

The additional memory devices 506, 508, and 510 can store data, programs, instructions, database queries in text or compiled form, and any other information that may be needed to operate a computer. The memories 506, 508 and 510 can be implemented as computer-readable media (integrated or removable), such as a memory card, disk drive, compact disk (CD), or server accessible over a network. In certain examples, the memories 506, 508 and 510 can comprise text, images, video, and/or audio, portions of which can be available in formats comprehensible to human beings. Additionally or alternatively, the system 500 can access an external data source or query source through the communication interface 512, which can communicate with the system bus 502 and the communication link 514.

In operation, the system 500 can be used to implement one or more parts of a system, such as that illustrated in FIGS. 1 and 2. Computer executable logic for implementing the system resides on one or more of the system memory 506, and the memory devices 508 and 510 in accordance with certain examples. The processing unit 504 executes one or more computer executable instructions originating from the system memory 506 and the memory devices 508 and 510. The term “computer readable medium” as used herein refers to a medium that participates in providing instructions to the processing unit 504 for execution. This medium may be distributed across multiple discrete assemblies all operatively connected to a common processor or set of related processors.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes, but is not limited to portable or fixed storage devices, optical storage devices, wireless channels, and/or various other storage mediums capable of storing that contain or carry instruction(s) and/or data.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on.

1. A method comprising:
receiving a first collected data set about the first user comprising tone information from a spoken communication between a first user and a second user
inputting the first collected data set to a machine learning model; and
providing a clinical parameter representing a mental state of the first user from the machine learning model; and
providing at least one prompt to the second user, wherein said at least one prompt is selected from the group consisting of a suggested phrase, a suggested sentence, and a suggested topic of conversation.

2. The method of claim 1, wherein the suggested phrase, the suggested sentence, and the suggested topic of conversation includes the suggested phrase and the selected sentence, and
wherein the method further comprises receiving a response from the second user selecting the one of the suggested phrase and the suggested sentence; and
playing audio representing the one of the suggested phrase and the suggested sentence to the first user.

3. The method of claim 2, additionally comprising a virtual environment user interface wherein each of the first user and the second user are represented as avatars within the virtual environment, and
animating the avatar associated with the second user to perform a gesture associated with the suggested phrase and the suggested sentence while playing the audio.

4. The method of claim 3 further comprising: providing the spoken communication to a voice recognition system to generate text representing the spoken communication; and
providing the text representing the spoken communication to the second user.

5. The method of claim 4, the method further comprising providing the text representing the spoken communication to a third user, the third user further receiving a text representing a spoken communication by a fourth user.

6. The method of claim 2, wherein the first user has an associated score generated by performing activities within the virtual environment, one of an associated score of the first user and a parameter derived from the associated score being displayed to the second user when interacting with the first user.

7. The method of claim 6, wherein the associated score is based on each of performing activities within the virtual environment and the collected data.

8. The method of claim 2, wherein the machine learning model additionally receives metadata reflecting a frequency with which the first user accesses the virtual environment and an amount of time for which the first user accesses the virtual environment.

9. The method of claim 1, wherein the machine learning model additionally receives answers to a survey provided to the first user.

10. The method of claim 9, further comprising providing the survey to the first user in response to a second clinical parameter generated from a second collected data set.

11. A system comprising an input device suitable for hosting interactions between a first user and a second user in a virtual environment on a server, and a helper support system;
wherein the input device includes a microphone suitable for receiving spoken communication from the first user; and
wherein the server comprises a network interface, a processor, and a non-transitory computer medium that stores executable instructions that, when executed by the processor, provides at least one prompt to the second user wherein said at least one prompt is selected form the group consisting of a suggested phrase, a suggested sentence, and a suggested topic of conversation,
wherein said helper support system comprises a data collection component that is suitable for collecting data about the first user when the first user interacts with the second user and producing collected data comprising tone information from the spoken communication, and
a machine learning model suitable for receiving the collected data and selecting the prompt provided to the second user.

12. The system of claim 11, the input device comprises at least one of a handheld sensor and a wearable sensor, and wherein the collected data comprises data received from the at least one handheld sensor and the wearable sensor.

13. The system of claim 12, wherein the data from the at least one handheld sensor and the wearable sensor comprises physiological data representing the first user.

14. The system of claim 12, wherein the data from the at least one handheld sensor and the wearable sensor comprises an estimated location of a gaze of the first user within the virtual environment and a location of the user within the virtual environment.

15. The system of claim 11, wherein the system further comprises a speaker, and the executable instructions further providing, upon execution by the processor, a text-to-speech component that plays audio representing the one of the suggested phrase and the suggested sentence to the first user.

16. The system of claim 11, wherein the first user is suffering from at least one specific psychological issue selected from the group consisting of anxiety, depression, addiction, stress, and grief.

17. (canceled)

18. A method comprising:
collecting data from a spoken communication from a first user to a second user in a virtual environment about the first user as the first user interacts with the second user, wherein the collected data comprises tone information from the spoken communication of the first user;
outputting a clinical parameter comprising a mental state of the first user from applying the collected data to a machine learning model; and
providing at least two prompts to the second user, wherein said prompts comprise at least one of a suggested phrase or a suggested sentence;
receiving a response from the second user selecting the one of the suggested prompts; and
playing audio representing the selected prompt to the first user.

19. The method of claim 18, wherein each of the first user and the second user are represented as avatars within the virtual environment, the method further comprising animating the avatar associated with the second user to move a mouth of the avatar and perform a gesture associated with the suggested phrase and the suggested sentence while playing the audio.

20. The method of claim 18, further comprising providing a survey to the first user in response to the clinical parameter.
